SEQUENCE LISTING (30 sequences)

SEQ ID NO: 1 - RNA, 67 nt, MERS-CoV
  Leader (1)..(67): Leader sequence
gauuuaagugaauagcuuggcuaucucacuuccccucguucucuugcagaacuuugauuuuaacgaa

SEQ ID NO: 2 - RNA, 71 nt, HCoV-229E
  Leader (1)..(71): Leader sequence
acuuaaguaccuuaucuaucuacagauagaaaaguugcuuuuuagacuuugugucuacuuuucucaacuaa

SEQ ID NO: 3 - RNA, 70 nt, HCoV-OC43
  Leader (1)..(70): Leader sequence
gauugugagcgauuugcgugcgugcaucccgcuucacugaucucuuguuagaucuuuuuguaaucuaaac

SEQ ID NO: 4 - RNA, 72 nt, HCoV-NL63
  Leader (1)..(72): Leader sequence
cuuaaagaauuuuucuaucuauagauagagaauuuucuuauuuagacuuugugucuacucuucucaacuaaa

SEQ ID NO: 5 - RNA, 71 nt, HCoV-HKU1
  Leader (1)..(71): Leader sequence
gaguuugagcgauugacguucguaccgucuaucagcuuacgaucucuugucagaucucauuaaaucuaaac

SEQ ID NO: 6 - DNA, 19 bp, Artificial Sequence
  Forward primer for MERS-CoV
agcttggctatctcacttc

SEQ ID NO: 7 - DNA, 23 bp, Artificial Sequence
  Reverse primer for MERS-CoV
agttcgttaaaatcaaagttctg

SEQ ID NO: 8 - DNA, 16 bp, Artificial Sequence
  Probe sequence for MERS-CoV
agactttgtgtctact

SEQ ID NO: 9 - DNA, 22 bp, Artificial Sequence
  Forward primer for HCoV-229E
ctacagatagaaaagttgcttt

SEQ ID NO: 10 - DNA, 21 bp, Artificial Sequence
  Reverse primer for HCoV-229E
ggtcgtttagttgagaaaagt

SEQ ID NO: 11 - DNA, 16 bp, Artificial Sequence
  Probe sequence for HCoV-229E
agactttgtgtctact

SEQ ID NO: 12 - DNA, 15 bp, Artificial Sequence
  Forward primer for HCoV-OC43
aaacgtgcgtgcatc

SEQ ID NO: 13 - DNA, 24 bp, Artificial Sequence
  Reverswe primer for HCoV-OC43
agattacaaaaagatctaacaaga

SEQ ID NO: 14 - DNA, 17 bp, Artificial Sequence
  Probe sequence for HCoV-OC43
cttcactgatctcttgt

SEQ ID NO: 15 - DNA, 26 bp, Artificial Sequence
  Forward primer for HCoV-NL63
ggagatagagaattttcttatttaga

SEQ ID NO: 16 - DNA, 20 bp, Artificial Sequence
  Reverse primer for HCoV-NL63
ggtttcgtttagttgagaag

SEQ ID NO: 17 - DNA, 16 bp, Artificial Sequence
  Probe sequence for HCoV-NL63
gtgtctactcttctca

SEQ ID NO: 18 - DNA, 17 bp, Artificial Sequence
  Forward primer for HCoV-HKU1
cgtaccgtctatcagct

SEQ ID NO: 19 - DNA, 24 bp, Artificial Sequence
  Reverse primer for HCoV-HKU1
gtttagatttaatgagatctgaca

SEQ ID NO: 20 - DNA, 14 bp, Artificial Sequence
  Probe sequence for HCoV-HKU1
acgatctcttgtca

SEQ ID NO: 21 - DNA, 45 bp, Artificial Sequence
  Forward primer for MERS-CoV 1 to 563 amplification
taatacgactcactatagggatttaagtgaatagcttggctatct

SEQ ID NO: 22 - DNA, 22 bp, Artificial Sequence
  Reverse primer for MERS-CoV 1 to 563 amplification
tgagcctctcaaccaggtatac

SEQ ID NO: 23 - DNA, 47 bp, Artificial Sequence
  Forward primer for HCoV-229E 1 to 563 amplification
taatacgactcactatagggacttaagtaccttatctatctacagat

SEQ ID NO: 24 - DNA, 19 bp, Artificial Sequence
  Reverse primer for HCoV-229E 1 to 563 amplification
ccaaccacccatgaagcat

SEQ ID NO: 25 - DNA, 40 bp, Artificial Sequence
  Forward primer for HCoV-OC43 1 to 563 amplification
taatacgactcactataggggattgtgagcgatttgcgtg

SEQ ID NO: 26 - DNA, 21 bp, Artificial Sequence
  Reverse primer for HCoV-OC43 1 to 563 amplification
tgcctctaaagacatacctaa

SEQ ID NO: 27 - DNA, 42 bp, Artificial Sequence
  Forward primer for HCoV-NL63 1 to 563 amplification
taatacgactcactatagggcttaaagaatttttctatctat

SEQ ID NO: 28 - DNA, 18 bp, Artificial Sequence
  Reverse primer for HCoV-NL63 1 to 563 amplification
atgagccaacctcgcaaa

SEQ ID NO: 29 - DNA, 41 bp, Artificial Sequence
  Forward primer for HCoV-HKU1 1 to 563 amplification
taatacgactcactataggggagtttgagcgattgacgttc

SEQ ID NO: 30 - DNA, 20 bp, Artificial Sequence
  Reverse primer for HCoV-HKU1 1 to 563 amplification
accctttaggaaaaccaccc

(19) United States
(12) Patent Application Publication
(10) Pub. No.: US 2018/0127836 A1
(43) Pub. Date: May 10, 2018

(54) IMPROVED COMPOSITIONS AND METHODS FOR DETECTION OF VIRUSES

(71) Applicant: EMERGING VIRAL DIAGNOSTIC LIMITED, Central (CN)

(72) Inventors: Fuk Woo Jasper CHAN, Hong Kong (CN); Man Lung YEUNG, Hong Kong (CN); Kai Wang Kelvin TO, Hong Kong (CN); Kwok Yung YUEN, Hong Kong (CN); Johnson Yiu-Nam LAU, Newport Beach, CA (US); Manson FOK, Hong Kong (CN)

(21) Appl. No.: 15/572,125

(22) PCT Filed: May 6, 2016
(86) PCT No.: PCT/US2016/031240

§ 371 Date: Nov. 6, 2017

Related U.S. Application Data
(60) Provisional application No. 62/158,490, filed on May 7, 2015.

(51) Int. Cl. C12Q 1/6888; C12Q 1/70; C12Q 1/6811; C12Q 1/6809; C12Q 1/686

(52) U.S. Cl. CPC C12Q 1/6888; C12Q 1/702; C12Q 1/6811; C12Q 1/6809; C12Q 2600/16; C12Q 2600/112; C12Q 2600/156; C12N 2770/20011; C12Q 1/686

(57) ABSTRACT

Highly conserved, short untranslated leader sequences have been identified in MERS-CoV and other human pathogenic Coronaviruses that provide the basis for highly sensitive and accurate assays for these viruses. Use of locked nucleic acids is shown to be useful in amplification reactions for these short sequences. RT-PCR using locked nucleic acids is shown to provide accurate detection of a variety of human pathogen Coronaviruses present at 10 copies per reaction or less.

This application claims the benefit of U.S. Provisional Application No. 62/158,490, filed May 7, 2015, which is hereby incorporated in its entirety.

### FIELD OF THE INVENTION

The field of the invention is detection of viruses, preferably RNA viruses and especially coronaviruses and influenza viruses.

### BACKGROUND OF THE INVENTION

The background description includes information that may be useful in understanding the typical invention. It is not an admission that any of the information provided herein is prior art or relevant to the presently claimed invention, or that any publication specifically or implicitly referenced is prior art.

Throughout history, viruses (for example coronaviruses, or CoVs) have repeatedly crossed species barriers, and some have emerged as important human pathogens (Lau et al, J Virol 85:11325-11337, 2011). Their clinical significance and impact on public health are best exemplified by the recent epidemics of SARS in 2003 and MERS since 2012 (Cheng et al, Clin Microbiol Rev 20:660-694, 2007; Chan et al, Clin Microbiol Rev 28:465-522, 2015). All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply. Highly sensitive and specific laboratory diagnostic tests are essential for case identification, contact tracing, animal source finding, and rationalization of infection control measures for the control of emerging viral outbreaks.

Isolation of viruses in cell culture is the gold standard of laboratory diagnosis. Unfortunately some important emerging pathogens, including CoVs, are difficult to culture in cell lines. In addition culture of many viruses requires then use of biosafety level-3 facilities, which are not routinely available in most clinical laboratories (Chan et al, J Infect Dis 207:1743-1752, 2013). As illustrated by the evolving MERS epidemic, molecular diagnosis by real-time RT-PCR has become the method of choice for establishing laboratory diagnosis of CoV infections and is widely available in most clinical microbiology laboratories (Corman et al, Euro Surveill 17. pii: 20285, 2012; Corman et al, Euro Surveill 17.pii: 20334, 2012). It is generally accepted that gene targets which are highly abundant in the CoV genome are useful RT-PCR targets. This principle is well illustrated by previously established RT-PCR assays that target the abundantly expressed N gene of CoVs which is located at the 3′ terminus of the genome (Cheng et al, Clin Microbiol Rev 20:660-694, 2007; Chan et al, Clin Microbiol Rev 28:465-522, 2015).

Six coronaviruses (CoVs) are known to cause human infection (Chan et al, J Infect 65:477-489, 2012; Chan et al, J Formos Med Assoc 112:372-381, 2013). Human CoV (HCoV)-229E, HCoV-OC43, HCoV-NL63, and HCoV-HKU1 predominantly cause mild upper respiratory tract infections, while severe acute respiratory syndrome CoV (SARS-CoV) and the novel Middle East respiratory syndrome CoV (MERS-CoV) frequently cause severe pneumonia with extrapulmonary manifestations. Most CoVs are, however, notoriously difficult to culture in cell lines (5). For MERS-CoV which replicates rapidly in a wide range of cell lines and SARS-CoV which grows in selected cell lines, the requirement of biosafety level-3 facility limits the practical application of cell culture (Chan et al, J Infect Dis 207:1743-1752, 2013).

Immunologic assays to detect specific neutralizing antibodies in serum samples taken at the acute and convalescent phases spaced 14 to 21 days apart can also provides evidence of infection. However, the need of convalescent samples and issues with false-positive results from cross-reactivity with other CoVs limit their use in the acute setting (Woo et al, J Clin Microbiol 42:2306-2309, 2004). Antigen detection assays are also available for some of these CoVs, but the overall sensitivity is inferior to that of molecular assays such as reverse transcription-polymerase chain reaction (RT-PCR) (Lau et al, J Clin Virol 45:54-60, 2005; Song et al, J Clin Microbiol 53:1178-1182, 2015). With the increasing availability of molecular diagnostic facilities and expertise in clinical microbiology laboratories worldwide, RT-PCR has become the test of choice for establishing the diagnosis of many viral infections (Corman et al, Euro Surveill 17. pii: 20285, 2012; de Sousa et al, J Clin Virol 59:4-11, 2014).

Traditionally, the preferred targets of RT-PCR assays are genes that are conserved and/or abundantly expressed from the viral genome (Sridhar et al, J Mol Diagn pii: S1525-1578(15)00038-0, 2015). For CoVs, the most commonly employed targets include the structural nucleocapsid (N) and spike (S) genes, and the non-structural RNA dependent RNA polymerase (RdRp) and replicase ORF1a/b genes. Recently, other unique non-coding genome regions not typical in related CoVs have also been utilized to develop RT-PCR for the emerging MERS-CoV. Currently, the World Health Organization recommends using the upE assay (regions upstream of the envelope [E] gene) for laboratory screening of suspected MERS cases, followed by confirmation with either the ORF1a or ORF1b assays.

Notably, a number of single nucleotide mismatches at different positions included in the upE assay forward primer and probe have been detected in recent strains of MERS-CoV and may affect the sensitivity of the assay (Corman et al, J Clin Virol 60:168-171, 2014). In addition, RT-PCR assay developed for detection of CoVs to date take considerable time and lack the sensitivity and/or specificity for full implementation as clinical tests.

Thus, there is still a need for rapid and accurate methods for the identification of pathogenic viruses that are suitable for clinical use.

### SUMMARY OF THE INVENTION

The inventive subject matter provides apparatus, systems and methods in which an RNA virus, for example a coronavirus (CoV), can be detected. In embodiments of the inventive concept a highly conserved RNA sequence that is represented in high copy numbers in infected cells is identified. Such a sequence can represent 3%, 3.5%, 4%, 4.5%, 5%, 7.5%, 10% or more of the viral RNA associated with an infected cell. The highly conserved RNA sequence can be an untranslated sequence, for example a sequence corresponding to a leader sequence. Such a leader sequence can be a 5′ untranslated region positioned upstream of a transcription regulatory sequence. Such target sequences can range in length from 30 to 200 nucleotides, from 40 to 100 nucleotides, or from 60 to 90 nucleotides in length.

In some embodiments the target sequence is present in sufficient numbers to permit detection by direct hybridization to a probe and/or a capture sequence (for example, using either two-strand/duplex or three-strand/triplex formation) without the use of an intervening amplification step. Alternatively, amplification-based methods such as PCR, reverse transcription polymerase chain reaction (RT-PCR), ligase chain reaction, and so on can be utilized to amplify the target sequence to facilitate a detection step. In some embodiments detection can take place during amplification to permit real time detection. In other embodiments detection can take place following amplification to permit end point detection.

Amplification reactions can be carried out using non-naturally occurring nucleotides, for example LNAs, in order to improve the performance of such amplification-based methods with relatively short nucleotide sequences. Similarly, hybridization step can be carried out using nucleic acid sequences that incorporate non-naturally occurring nucleotides. Other suitable non-naturally occurring nucleic acids include PNAs and xeno nucleic acids.

In some embodiments mismatches relative to a target sequence can be incorporated into probe sequences and/or primer sequences utilized in such assays. For example, between 5% and 50% of the nucleotides of a probe sequence or primer sequence can be mismatches for the corresponding nucleotides of a target sequence.

### FIGURES

FIG. 1 provides a schematic diagram of a portion of the MERS-CoV genome. The leader sequence of the 5′-untranslated region is enlarged to show the abundance of small RNA sequences. The percentages of mapped small RNA sequence reads at the leader sequence, ORF1a, S, and N gene regions are quantified and shown. FIG. 1 also shows the sequence of the leader portion of the MERS-CoV genome (SEQ ID NO. 1), and additionally provides typical 70 to 72 nucleotide leader sequences of HCoV-229E (SEQ ID NO. 2), HCoV-OC43 (SEQ ID NO. 3), HCoV-NL63 (SEQ ID NO. 4), and HCoV-HKU1(SEQ ID NO. 5).

FIGS. 2A to 2J depict typical RT-PCR results for amplification of leader sequences of a variety of CoV human pathogens using primers and probes of the inventive concept. FIG. 2A shows typical fluorescence vs time plots for RT-PCR of MERS-CoV provided at 10⁸ to 10¹ copies per reaction (cpr). FIG. 2B shows a typical dose/response curve for RT-PCR of MERS-CoV using a primer/probe set of the inventive concept. FIG. 2C shows typical fluorescence vs time plots for RT-PCR of HCoV-229E provided at 10⁸ to 10¹ copies per reaction (cpr). FIG. 2D shows a typical dose/response curve for RT-PCR of HCoV-229E using a primer/probe set of the inventive concept. FIG. 2E shows typical fluorescence vs time plots for RT-PCR of HCoV-OC43 provided at 10⁸ to 10¹ copies per reaction (cpr). FIG. 2F shows a typical dose/response curve for RT-PCR of HCoV-NL63 using a primer/probe set of the inventive concept. FIG. 2G shows typical fluorescence vs time plots for RT-PCR of HCoV-OC43 provided at 10⁸ to 10¹ copies per reaction (cpr). FIG. 2H shows a typical dose/response curve for RT-PCR of HCoV-NL63 using a primer/probe set of the inventive concept. FIG. 2I shows typical fluorescence vs time plots for RT-PCR of HCoV-HKU1 provided at 10⁸ to 10¹ copies per reaction (cpr). FIG. 2J shows a typical dose/response curve for RT-PCR of HCoV-HKU1 using a primer/probe set of the inventive concept.

### DETAILED DESCRIPTION

The following description includes information that may be useful in understanding the typical invention. It is not an admission that any of the information provided herein is prior art or relevant to the presently claimed invention, or that any publication specifically or implicitly referenced is prior art.

The inventors have identified a relatively short untranslated region located 5′ upstream to a transcription regulatory sequence that is, surprisingly, both overexpressed and highly conserved in coronaviruses. When used as a target for RT-PCR (particularly in conjunction with the use of LNAs to at least partially offset the effects of short length) or similar analytical methods such a sequence supports assays for coronaviruses with improved sensitivity and/or specificity for such viruses relative to approaches used in the prior art.

One should appreciate that the disclosed techniques provide many advantageous technical effects including improved accuracy, improved sensitivity, and/or reduced time to result relative to prior art methods for detection of viruses.

Based on the discussed discoveries and the described in further detail below, the inventors contemplate that reagents, kits, and methods of the inventive concept are applicable to any viral species, including RNA viruses. Suitable RNA viruses include coronaviruses, (i.e. members of the genera Alphacoronavirus, Betacoronavirus, Gammacoronavirus, and/or Deltacoronavirus, inclusive of species that are causative for SARS and MERS), Astroviridae, Caliciviridae, Picornaviridae, Flaviviridae, Retroviridae, Togaviridae, Arenaviridae, Bunyaviridae, Filoviridae, Orthomyxoviridae, Paramyxoviridae, Rhabdoviridae, and/or Reoviridae. Influenza viruses, for example influenza A and/or influenza B, are also considered. In a preferred embodiment of the inventive concept, reagents, kits, and methods of the inventive concept are directed to a coronavirus (CoV).

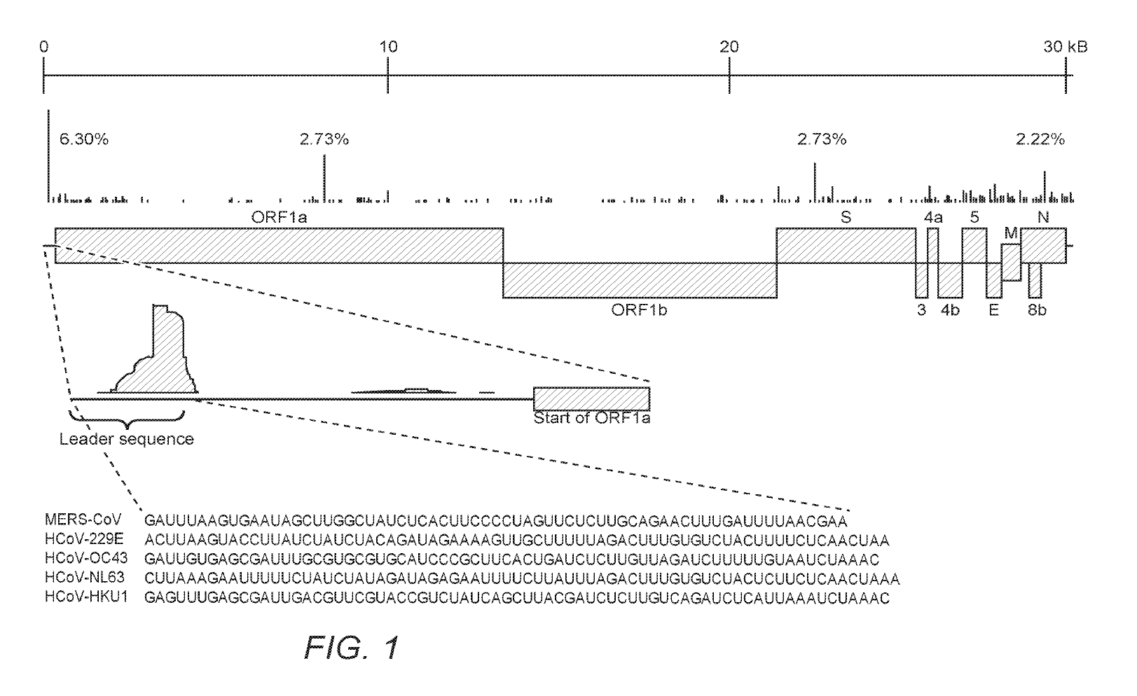

The single-stranded RNA genome of CoVs is around 26 to 31 kb in length and contains 5′-capped, 3′-polyadenylated, polycistronic RNA. In general, the genome arrangement follows the order of 5′-replicase (ORF1a/b)-structural protein genes (spike [S]-envelope [E]-membrane [M]-nucleocapsid [N])-poly(A)-3′ with the exception of lineage A βCoVs which have the characteristic S-like hemagglutinin-esterase (HE) gene located between the replicase and S genes. Leader sequences of about 60 to 90 nucleotides in length can be found at the 5′-UTR upstream from the transcription regulatory sequence in the genomes and at the subgenomic RNAs of all CoVs; the function of these leader sequences is, however, poorly understood. In a typical study, a small RNA sequence data analysis identified a 67-nucleotide leader sequence that is, surprisingly, the most abundantly expressed gene region in the MERS-CoV genome (FIG. 1).

FIG. 1 shows a schematic diagram representing a MERS-COV genome. In FIG. 1 the leader sequence associated with the 5′ untranslated region is enlarged to show the abundance of small RNA sequences associated with this region. The percentages shown represent the percentage of small RNA sequences associated with the leader sequence, ORF1a, S, and N genes for this virus. Other studies have shown that similar sequences are present in other coronaviruses. FIG. 1 additionally shows the sequences of 70 to 72 nucleotide regions that present abundant RNA that are found in other human coronaviruses, such as HCoV-229E, HCoV-OC43, HCoV-NL63, and HCoV-HKU1). Suitable small RNA sequences can have lengths of 30 to 200 nucleotides, from 40 to 100 nucleotides, or from 60 to 90 nucleotides. The Inventors contemplate that similar sequences are present in other human pathogenic virus species, including influenza viruses, Alphacoronavirus, Betacoronavirus, Gammacoronavirus, and/or Deltacoronavirus (inclusive of species that are causative for SARS and MERS), Astroviridae, Caliciviridae, Picornaviridae, Flaviviridae, Retroviridae, Togaviridae, Arenaviridae, Bunyaviridae, Filoviridae, Orthomyxoviridae, Paramyxoviridae, Rhabdoviridae, and/or Reoviridae

The inventors have found that the leader sequence are a valuable diagnostic target not only for MERS-CoV, and similar leader sequences can serve as diagnostic targets for other currently circulating HCoVs which similarly possess leader sequences. Similar leader sequences in other viral species, including Astroviridae, Caliciviridae, Picornaviridae, Flaviviridae, Retroviridae, Togaviridae, Arenaviridae, Bunyaviridae, Filoviridae, Orthomyxoviridae, Paramyxoviridae, Rhabdoviridae, Reoviridae, and/or Influenza viruses, can similarly provide diagnostic targets for infection with such species.

The relatively short length of such leader sequences can be a barrier to detection and/or amplification. The Inventors have found that the use of non-naturally occurring nucleic acids (for example, in probe sequences, primer sequences, and/or hybridization/capture nucleic acid sequences) can offset this effect. Suitable non-naturally occurring nucleic acids include locked nucleic acids (LNA), peptide nucleic acids (PNA), and xeno nucleic acids. For example, an LNA-containing probe sequence can be utilized in a real-time RT-PCR LNA assay that targets the leader sequences of human pathogenic CoVs. Such an LNA-containing probe sequence includes one or more nucleic acid analogs that provide increased hybridization affinity (relative to native DNA and RNA) towards complementary DNA and RNA sequences, while also providing efficient mismatch discrimination. These properties are associated with an increased melting temperature of the hybrids formed from such oligonucleotides, which allows the application of shorter probes when LNA rather than DNA nucleotides are used in the nucleic acid amplification assays. Such LNA-containing probes can include a single LNA, two LNAs, 3 LNAs, or more than 3 LNAs. In some embodiments 0.5%, 1%, 2%, 3%, 4%, 5% or more of the nucleic acids in a primer, probe, or hybridization/capture nucleic acid sequence can be non-naturally occurring nucleic acids.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

It should be appreciated that a wide variety of detection methodologies are suitable for methods of the inventive concept. For example, samples can be assayed by direct hybridization of polynucleotides obtained from infected cells or samples containing infected cells without an intervening amplification step (for example amplification using an exogenous polymerase, such as PCR or RT-PCR). Such approaches are relatively simple to implement and are less subject to contamination. Suitable direct hybridization methods include capture of the target sequence using solid-phase conjugated capture sequence (for example, a nucleic acid microarray, nucleic acid modified microwell plate, nucleic acid modified bead, or nucleic acid conjugated microparticles) and detection of hybrid formation. Hybrid formation can be detected by any suitable means. Suitable methods for hybrid detection include detection of an observable label associated with or dissociated from a hybrid (for example, a fluorescent label, a colorimetric label, a spin label, a mass label, and/or an affinity label), changes in FRET behavior of fluorophore-bearing members of the hybrid, selective dye binding (for example, major or minor groove binding dyes), UV absorbance, and changes in refractive index. Alternatively, hybrid formation can be detected using a separation technique, such as electrophoresis (for example, capillary or gel electrophoresis). Such techniques can be relatively technically simple and quantitative. In addition, use of sequence encoding (for example, by position within a microarray or by fluorescence properties of a set of microparticles) can simplify simultaneous characterization of a polynucleotide obtained from a sample against multiple probe sequences, and support multiplex testing. Such techniques may not be suitable, however, for situations where the target virus may be present in low abundance.

Alternatively, in other embodiments polynucleotides from infected cells or samples containing infected cells can be characterized using amplification methods that employ exogenous polymerases, such as DNA polymerases and/or reverse transcriptases, which can be obtained from thermophilic organisms (thereby supporting thermal cycling amplification methods). Suitable amplification methods include PCR, nested PCR, RT-PCR, transcription mediated amplification (TMA), strand displacement amplification (SDA), and nucleic acid based sequence amplification (NASBA).

Detection of hybridization events and/or the formation of amplification products can be facilitated by incorporating detectable tags into probe and/or primer sequences. Suitable detectable tags include fluorophores, chromophores, spin labels, radioactive isotopes, affinity epitopes (for example, biotin or digoxigenin), and/or mass tags. Detection methodologies utilized depend upon the incorporated tag. For example, fluorophores can be detected by fluorescence measurement, characterization of FRET, fluorescence quenching, and/or fluorescence anisotropy, which can in turn be measured in a static sample or in a sample undergoing separation (for example, by capillary electrophoresis). Mass tags can be characterized by subjecting method products to mass spectroscopy. Affinity epitopes can be detected by complex formation with a corresponding affinity-directed molecule, for example avidin, streptavidin, and/or epitope-specific antibodies or antibody fragments. Such affinity-directed molecules can include directly observable detection moieties (for example fluorophores, lumiphores, and/or chromophores) or indirectly observable detection moieties (for example luciferase or an enzyme with a chromomeric or fluorogenic substrate).

In a preferred embodiment of the inventive concept, RT-PCR is used. The analytical sensitivities and specificities of a typical real-time RT-PCR LNA assay were found to be excellent. A limit of detection of 5 to 10 RNA copies/reaction (in vitro RNA transcripts) and 5.62×10⁻² TCID₅₀/ml (genomic RNA) for the MERS-CoV-LS assay (see Table 1) were comparable with those for the other assays currently recommended for screening and/or confirmation of MERS by the World Health Organization.

TABLE 1

| Predicted no. of RNA | No. of positive tests/no. transcript replicates (4%) | | | | |
|---|---|---|---|---|---|
| copies/reaction | MERS-CoV-LS | HCoV-229E-LS | HCoV-OC43-LS | HCoV-NL63-LS | HCoV-HKU1-LS |
| 20 | 10/10 (100) | 10/10 (100) | 10/10 (100) | 10/10 (100) | 10/10 (100) |
| 10 | 10/10 (100) | 10/10 (100) | 10/10 (100) | 10/10 (100) | 10/10 (100) |
| 5 | 6/10 (60) | 6/10 (60) | 9/10 (90) | 10/10 (100) | 10/10 (100) |

For comparison, the prior art ORF1b assay for MERS CoV has a least optimal limit of detection of 64 RNA copies/reaction. CoV real time RT-PCR LNA assays of the inventive concept showed no cross-reactivity among the individual CoVs and with other common respiratory viruses including influenza A and B viruses, parainfluenza virus types 1 to 4, rhinovirus/enterovirus, respiratory syncytial virus, and human metapneumovirus (see Table 2).

TABLE 2

| | | MERS- | HCoV- | HCoV- | HCoV- | HCoV- |
|---|---|---|---|---|---|---|
| Virus | Source | CoV-LS | 229E-LS | OC43-LS | NL63-LS | HKU1-LS |
| MERS-CoV | Laboratory strain | + | − | − | − | − |
| HCoV-229E | Clinical specimen | − | + | − | − | − |
| HCoV-229E | Laboratory strain | − | + | − | − | − |
| HCoV-OC43 | Clinical specimen | − | − | + | − | − |
| HCoV-OC43 | Laboratory strain | − | − | + | − | − |
| HCoV-NL63 | Clinical specimen | − | − | − | + | − |
| HCoV-HKU1 | Clinical specimen | − | − | − | − | + |
| IAV (H1N1) | Clinical specimen | − | − | − | − | − |
| IAV (H1N1) | Laboratory strain | − | − | − | − | − |
| IAV (H3N2) | Clinical specimen | − | − | − | − | − |
| IAV (H3N2) | Laboratory strain | − | − | − | − | − |
| IBV | Clinical specimen | − | − | − | − | − |
| IBV | Laboratory strain | − | − | − | − | − |
| PIF 1 | Clinical specimen | − | − | − | − | − |
| PIF 1 | Laboratory strain | − | − | − | − | − |
| PIF 2 | Clinical specimen | − | − | − | − | − |
| PIF 2 | Laboratory strain | − | − | − | − | − |
| PIF 3 | Clinical specimen | − | − | − | − | − |
| PIF 3 | Laboratory strain | − | − | − | − | − |
| PIF 4 | Clinical specimen | − | − | − | − | − |
| PIF 4 | Laboratory strain | − | − | − | − | − |
| RV/EV | Clinical specimen | − | − | − | − | − |
| RV/EV | Laboratory strain | − | − | − | − | − |
| RSV | Clinical specimen | − | − | − | − | − |
| RSV | Laboratory strain | − | − | − | − | − |
| hMPV | Clinical specimen | − | − | − | − | − |
| hMPV | Laboratory strain | − | − | − | − | − |

Abbreviations: +, positive. −, negative. EV, enterovirus. HCoV, human coronavirus, IAV, influenza A virus: IBV, influenza B virus; hMPV, human metapneumovirus, MERS-CoV, Middle East respiratory syndrome coronavirus: PIF, parainfluenza virus, RSV, respiratory syncytial virus; RV, rhinovirus.

An evaluation of the performance of a CoV real-time RT-PCR LNA assay of the inventive concept and compared it to the commercial ResPlex II® assay was also performed, using an in-use evaluation of 229 nasopharyngeal aspirates. The ResPlex II® assay is a commercially available multiplex PCR assay which detects 18 respiratory viruses (including HCoV-229E, HCoV-OC43, HCoV-NL63, and HCoV-HKU1), and is commonly employed for laboratory diagnosis of viral respiratory tract infections. A CoV real-time RT-PCR LNA assay of the inventive concept identified samples as positive for HCoVs with viral loads ranging from 13.7 RNA copies/reaction to 3.86×10⁸ RNA copies/reaction in all 49 (100%) nasopharyngeal aspirates that tested positive for HCoVs by ResPlex II® (see Tables 3A and 3B).

TABLE 3A

| | | CoV | | | | |
|---|---|---|---|---|---|---|
| | | real-time | | | | |
| | | LNART- | | | | |
| | | PCR result | | | | |
| | ResPlex II | RNA | | | | |
| | bead | copies/ | HCoV- | HCoV- | HCoV- | HCoV- |
| Sample no. | count | reaction) | 229E-LS | OC43-LS | NL63-LS | HKU1-LS |
| HCoV-229E-1 | 3.60 × 10⁸ | 1.64 × 10⁸ | + | − | − | − |
| HCoV-OC43-1 | 6.03 × 10⁸ | 4.53 × 10⁸ | − | + | − | − |
| HCoV-OC43-2 | 5.55 × 10⁸ | 7.62 × 10⁸ | − | + | − | − |
| HCoV-OC43-3 | 5.15 × 10⁸ | 3.29 × 10⁸ | − | + | − | − |
| HCoV-OC43-4 | 4.96 × 10⁸ | 2.56 × 10⁸ | − | + | − | − |
| HCoV-OC43-5 | 4.50 × 10⁸ | 1.24 × 10⁸ | − | + | − | − |
| HCoV-OC43-6 | 4.25 × 10⁸ | 2.78 × 10⁸ | − | + | − | − |
| HCoV-OC43-7 | 3.71 × 10⁸ | 1.10 × 10⁸ | − | + | − | − |
| HCoV-OC43-8 | 3.06 × 10⁸ | 8.33 × 10⁸ | − | + | − | − |
| HCoV-OC43-9 | 3.05 × 10⁸ | 3.02 × 10⁸ | − | + | − | − |
| HCoV-OC43-10 | 2.91 × 10⁸ | 1.37 × 10⁸ | − | + | − | − |
| HCoV-OC43-11 | 2.84 × 10⁸ | 1.21 × 10⁸ | − | + | − | − |
| HCoV-OC43-12 | 2.72 × 10⁸ | 6.09 × 10⁸ | − | + | − | − |
| HCoV-OC43-13 | 2.43 × 10⁸ | 3.86 × 10⁸ | − | + | − | − |
| HCoV-OC43-14 | 2.19 × 10⁸ | 1.12 × 10⁸ | − | + | − | − |
| HCoV-OC43-15 | 8.79 × 10⁸ | 2.57 × 10⁸ | − | + | − | − |
| HCoV-OC43-16 | 3.39 × 10⁸ | 8.67 × 10⁸ | − | + | − | − |
| HCoV-OC43-17 | 1.54 × 10⁸ | 1.89 × 10⁸ | − | + | − | − |
| HCoV-NL63-1 | 6.00 × 10⁸ | 1.69 × 10⁸ | − | − | + | − |
| HCoV-NL63-2 | 5.65 × 10⁸ | 9.68 × 10⁸ | − | − | + | − |
| HCoV-NL63-3 | 5.46 × 10⁸ | 1.00 × 10⁸ | − | − | + | − |
| HCoV-NL63-4 | 5.09 × 10⁸ | 4.71 × 10⁸ | − | − | + | − |
| HCoV-NL63-5 | 5.09 × 10⁸ | 3.54 × 10⁸ | − | − | + | − |
| HCoV-NL63-6 | 5.07 × 10⁸ | 5.88 × 10⁸ | − | − | + | − |
| HCoV-NL63-7 | 4.84 × 10⁸ | 2.54 × 10⁸ | − | − | + | − |

TABLE 3B

| | | CoV | | | | |
|---|---|---|---|---|---|---|
| | | real-time | | | | |
| | | LNART- | | | | |
| | | PCR result | | | | |
| | ResPlex II | RNA | | | | |
| | bead | copies/ | HCoV- | HCoV- | HCoV- | HCoV- |
| Sample no. | count | reaction) | 229E-LS | OC43-LS | NL63-LS | HKU1-LS |
| HCoV-NL63-8 | 4.80 × 10⁸ | 7.44 × 10⁸ | − | − | + | − |
| HCoV-NL63-9 | 4.63 × 10⁸ | 2.01 × 10⁸ | − | − | + | − |
| HCoV-NL63-10 | 4.40 × 10⁸ | 4.34 × 10⁸ | − | − | + | − |
| HCoV-NL63-11 | 4.08 × 10⁸ | 9.49 × 10⁸ | − | − | + | − |
| HCoV-NL63-12 | 3.92 × 10⁸ | 3.22 × 10⁸ | − | − | + | − |
| HCoV-NL63-13 | 3.64 × 10⁸ | 7.30 × 10⁸ | − | − | + | − |
| HCoV-NL63-14 | 3.34 × 10⁸ | 2.34 × 10⁸ | − | − | + | − |
| HCoV-NL63-15 | 3.21 × 10⁸ | 1.35 × 10⁸ | − | − | + | − |
| HCoV-NL63-16 | 3.07 × 10⁸ | 3.47 × 10⁸ | − | − | + | − |
| HCoV-NL63-17 | 2.98 × 10⁸ | 8.27 × 10⁸ | − | − | + | − |
| HCoV-NL63-18 | 2.95 × 10⁸ | 6.67 × 10⁸ | − | − | + | − |
| HCoV-NL63-19 | 2.57 × 10⁸ | 4.56 × 10⁸ | − | − | + | − |
| HCoV-NL63-20 | 2.21 × 10⁸ | 9.33 × 10⁸ | − | − | + | − |
| HCoV-NL63-21 | 1.88 × 10⁸ | 3.25 × 10⁸ | − | − | + | − |
| HCoV-NL63-22 | 1.44 × 10⁸ | 1.29 × 10⁸ | − | − | + | − |
| HCoV-NL63-23 | 1.28 × 10⁸ | 1.75 × 10⁸ | − | − | + | − |
| HCoV-NL63-24 | 1.18 × 10⁸ | 1.21 × 10⁸ | − | − | + | − |
| HCoV-NL63-25 | 2.63 × 10⁸ | 2.38 × 10⁸ | − | − | + | − |
| HCoV-NL63-26 | 1.50 × 10⁸ | 1.01 × 10⁸ | − | − | + | − |
| HCoV-NL63-27 | 1.07 × 10⁸ | 9.20 × 10⁸ | − | − | + | − |
| HCoV-HKU1-1 | 3.54 × 10⁸ | 4.42 × 10⁸ | − | − | − | + |
| HCoV-HKU1-2 | 3.31 × 10⁸ | 1.04 × 10⁸ | − | − | − | + |
| HCoV-HKU1-3 | 1.74 × 10⁸ | 2.60 × 10⁸ | − | − | − | + |
| HCoV-HKU1-4 | 1.27 × 10⁸ | 1.94 × 10⁸ | − | − | − | + |

Moreover, a CoV real-time RT-PCR LNA assay identified HCoVs in an additional 2.2% of nasopharyngeal aspirates that initially tested negative by ResPlex II® (possibly due to low viral loads of around 10 to 100 RNA copies/reaction). Overall, these results demonstrate that CoV real-time RT-PCR LNA assays of the inventive concept are highly sensitive and specific. It is should be appreciated that ResPlex II® and other multiplex PCR assays may be inferior to monoplex PCR assays for HCoVs and other respiratory viruses such as influenza A viruses. This relatively poor sensitivity can limit the application of such multiplex PCR assays for the detection of future emerging CoVs and avian influenza A viruses (which are potential pandemic agents that have significant public health impact if a case is misdiagnosed).

The inventors have demonstrated that small-RNA-Seq data analysis is helpful in the selection of optimal gene targets for the development of molecular diagnostic assays and should be considered for other emerging and circulating pathogenic viruses. The application of LNA probes permits the use of relatively short sequences, such as the leader sequence at the 5′-UTR of CoV genomes, as a diagnostic target. The inventors contemplate that such assays can be monoplex or multiplex assays, depending on the selection of primer sequence(s), probe sequence(s), and detectable tag(s). It should be appreciated that multiplex assays can have improved clinical utility relative to monoplex assays.

### Examples

Viruses and Clinical Specimens.

MERS-CoV (strain HCoV-EMC/2012), HCoV-229E, HCoV-OC43, HCoV-NL63, and HCoV-HKU1 were included in the exemplary studies. The MERS-CoV isolate was provided by R. Fouchier, A. Zaki, and colleagues. The isolate was amplified by one additional passage in Vero cells to make working stocks of the virus (5.62×10⁵ 50% tissue culture infective doses [TCID₅₀]/ml). All experimental protocols involving live MERS CoV followed the approved standard operating procedures of the biosafety level 3 facility. High-titer stocks of HCoV-229E, HCoV-OC43, and other respiratory viruses were prepared, and their TCID₅₀ values were determined using conventional methods. Attempts to culture HCoV-NL63 and HCoV-HKU1 were unsuccessful because of difficulties in culturing these using available cell lines. Virus positive clinical specimens (n=14) and laboratory strains (n=13) used for the validation of assays were obtained from archived clinical specimens at the clinical microbiology laboratory at Queen Mary Hospital. Total nucleic acid extracts of ResPlex II®-HCoV-positive (n=180) and ResPlex II®-HCoV-negative (n=49) respiratory clinical specimens were prepared according to the manufacturer's instructions using the QIAamp MinElute Virus Spin Kit®. A total of 243 fresh or frozen nasopharyngeal aspirates collected between 1 Jan. 2012 to 31 Oct. 2014 from 243 patients who were managed in either Queen Mary Hospital or Hong Kong Sanatorium and Hospital for upper and/or lower respiratory symptoms were included in the study.

Determination of the Most Abundantly Expressed Sequence in the MERS-CoV Genome by Small-RNA Sequence Data Analysis.

Calu-3 cells were inoculated with 3 logs TCID₅₀/ml MERS-CoV for 1 hour at 37° C. in triplicate. Unbound viruses were washed away with phosphate buffered saline (PBS). Total RNAs from the infected cells were harvested using EZ1 virus Mini Kit v2.0® (Qiagen®) at 12 hour post-infection. After RNA quantification, 1 μg of RNA was reverse transcribed into cDNA using random hexamers for high-throughput Illumina® sequencing. Sequencing reads were trimmed by removal of adapter and low quality ends using Trimmomatic version 0.32®. The length of the clean reads ranged from 13 to 101 nucleotides. Reads shorter or equal to 40 nucleotides were retained for further mapping. A total of 1,943,705 paired end remaining reads were used to map onto the MERS-CoV genome to determine the abundance of individual small RNA using Bowtie2 version 2.1.0®.

Results of these studies are shown in FIG. 1. As shown in FIG. 1, a high incidence of relatively short RNAs have been found to be associated with an untranslated leader sequence located upstream of ORF1a of the MERS-CoV genome. Similar results, yielding useful leader sequences of 70 to 72 nucleotides in length, were found with other human pathogen CoVs, including HCoV-229E, HCoV-OC43, HCoV-NL63, and HCoV-HKU1). Sequences of these target leader sequences are also shown in FIG. 1. Surprisingly, these untranslated sequences are highly conserved and can serve as target sequences for CoV-specific assays. The Inventors contemplate that similarly conserved untranslated leader sequences are present other viral pathogens (such as those detailed above), and can similarly serve as targets for virus-specific assays. Such untranslated leader sequences can range in size from as short as 30 nucleotides to as long as 200 nucleotides or more.

Nucleic Acid Extraction.

Total nucleic acid extractions of clinical specimens and laboratory cell culture with virus strains were performed on 200 μl of sample using EZ1 virus Mini Kit v2.0® (Qiagen) according to the manufacturer's instructions. Extracts were stored at minus 70° C. or below until use.

Primers and Probes.

Primer and probe sets targeting the conserved and highly expressed 70 to 72 nucleotide portions of leader sequences in the 5′-UTR of MERS-CoV, HCoV-229E, HCoV-OC43, HCoV-NL63, and HCoV-HKU1 were designed and tested. Primer and probe sets were predicted to specifically amplify the corresponding CoV and having no major combined homologies with human, other human pathogenic CoVs or microbial genes on BLASTn analysis that would potentially produce false-positive test results. Dual labeled LNA hydrolysis probes were used to detect the small target regions and to increase specificity and sensitivity of the real-time RT-PCR LNA assays. Primer and probe sets with the best amplification performance of each virus were selected (see Table 4).

TABLE 4

| | Genome | Genome | Primer/ | | GenBank |
|---|---|---|---|---|---|
| Assay | target | location | probe | Sequence (5′ to 3′) | accession no. |
| MERS-CoV-LS | Leader | 14-32 | Forward | AGCTTGGCTATCTCACTTC | JX869059.2 |
| | sequence | 47-69 | Reverse | AGTTCGTTAAAATCAAAGTTCTG | |
| | | 34-47 | Probe | C+CT+CGT+T+CT+CT+TGC | |
| HCoV-229E-LS | Leader | 20-41 | Forward | CTACAGATAGAAAAGTTGCTTT | NC_002645.1 |
| | sequence | 57-75 | Reverse | ggTCGTTTAGTTGAGAAAAGT | |
| | | 44-59 | Probe | AGACT+T+TG+TG+TCT+A+CT | |
| HCoV-OC43-LS | Leader | 17-28 | Forward | aaaCGTGCGTGCATC | NC_005147.1 |
| | sequence | 43-66 | Reverse | AGATTACAAAAAGATCTAACAAGA | |
| | | 32-48 | Probe | C+TTCA+CTG+ATCT+C+T+TGT | |
| HCoV-NL63-LS | Leader | 23-46 | Forward | ggAGATAGAGAATTTTCTTATTTAGA | NC_005831.2 |
| | sequence | 60-77 | Reverse | ggTTTCGTTTAGTTGAGAAG | |
| | | 50-66 | Probe | TGTGT+C+TAC+T+C+TTCT+CA | |
| HCoV-HKU1-LS | Leader | 21-37 | Forward | CGTACCGTCTATCAGCT | NC_006577.2 |
| | sequence | 48-71 | Reverse | GTTTAGATTTAATGAGATCTGACA | |
| | | 39-52 | Probe | ACGA+T+CT+C+TTG+T+CA | |

Abbreviations:
HCOV, human coronavirus;
MERS-COV, Middle East respiratory syndrome coronavirus;
UTR, untranslated region.
Remarks:
Probes were labeled at the 5′ end with the reporter molecule 6-carboxyfluorescein (6-FAM) and at the 3′ end with Iowa Black FQ (Integrated DNA Technologies, Inc). Lowercase letters represent the additional bases added which is not from the original genome sequence. The letters following “+” represent LNA bases which are modified with an extra bridge connecting the 2′ oxygen and 4′ carbon. The bridge “locks” the ribose in the 3′-endo (North) conformations and significantly increases the hybridization properties of the probe.

For MERS-CoV-LS, the forward primer sequence is identified as SEQ ID NO. 6, the reverse primer sequence is identified as SEQ ID NO. 7, and the probe sequence is identified as SEQ ID NO. 8. For HCoV-229-E-LS, the forward primer sequence is identified as SEQ ID NO. 9, the reverse primer sequence is identified as SEQ ID NO. 10, and the probe sequence is identified as SEQ ID NO. 11. For HCoV-OC43-LS, the forward primer sequence is identified as SEQ ID NO. 12, the reverse primer sequence is identified as SEQ ID NO. 13, and the probe sequence is identified as SEQ ID NO. 14. For HCoV-NL63-LS, the forward primer sequence is identified as SEQ ID NO. 15, the reverse primer sequence is identified as SEQ ID NO. 16, and the probe sequence is identified as SEQ ID NO. 17. For CoV-HKU1-LS, the forward primer sequence is identified as SEQ ID NO. 18, the reverse primer sequence is identified as SEQ ID NO. 19, and the probe sequence is identified as SEQ ID NO. 20.

In Vitro RNA Transcripts for Making Positive Controls and Standards.

Target regions with flanking regions of 5′-UTR of each of the five CoVs and containing a T7 RNA polymerase promoter sequence (TAATACGACTCACTATAGGG) (SEQ OD NO. 13) at the 5′ end were amplified to generate in vitro transcribed RNA using MEGAscript T7® kit (Ambion) for the standards and limit of detection. The primers used are listed in Table 5.

TABLE 5

| | Genome | | |
|---|---|---|---|
| Assay | locations | Primer | Sequence (5′ to 3′) |
| MERS-CoV- | 1-563 | Forward | TAATACGACTCACTATAGG |
| LS | | | GATTTAAGTGAATAGCTTG |
| | | | GCTATCT |
| | | Reverse | TGAGCCTCTCAACCAGGTA |
| | | | TAC |
| HCoV-229E- | 1-563 | Forward | TAATACCACTCACTATAGG |
| LS | | | GACTTAAGTACCTTATCTA |
| | | | TCTACAGAT |
| | | Reverse | CCAACCACCCATGAAGCAT |
| HCoV-OC43- | 1-563 | Forward | TAATACGACTCACTATAGG |
| LS | | | GGATTGTGAGCGATTTGCG |
| | | | TG |
| | | Reverse | TGCCTCTAAAGACATACCT |
| | | | AA |
| HCoV-NL63- | 1-563 | Forward | TAATACGACTCACTATAGG |
| LS | | | GCTTAAAGAATTTTTCTAT |
| | | | CTAT |
| | | Reverse | ATGAGCCAACCTCGCAAA |
| HCoV-HKU1- | 1-563 | Forward | TAATACGACTCACTATAGG |
| LS | | | GGAGTTTGAGCGATTGACG |
| | | | TTC |
| | | Reverse | ACCCTTTAGGAAAACCACC |
| | | | C |

Remarks:
TAATACGACTCACTATAGGG: T7 RNA polymerase promoter sequence.

For MERS-CoV-LS, the forward primer sequence is identified as SEQ ID NO. 21 and the reverse primer sequence is identified as SEQ ID NO. 22. For HCoV-229E-LS, the forward primer sequence is identified as SEQ ID NO. 23 and the reverse primer sequence is identified as SEQ ID NO. 24. For HCoV-OC43-LS, the forward primer sequence is identified as SEQ ID NO. 25 and the reverse primer sequence is identified as SEQ ID NO. 26. For HCoV-NL63-LS, the forward primer sequence is identified as SEQ ID NO. 27 and the reverse primer sequence is identified as SEQ ID NO. 28. For CoV-HKC1-LS, the forward primer sequence is identified as SEQ ID NO. 29 and the reverse primer sequence is identified as SEQ ID NO. 30.

The PCR products were purified using the QIAquick® gel extraction kit (QIAgen). Each purified amplicon was mixed with 2 μl each of ATP, GTP, CTP, and UTP, 10× reaction buffer, and enzyme mix in a standard 20 μl reaction mixture. The reaction mixture was incubated at 37° C. for 4 hours, followed by addition of 1 μl of TURBO DNase®, and was further incubated at 37° C. for 15 minutes. The synthesized RNA was purified by phenol-chloroform extraction. The concentration of purified RNA was quantified by UV light absorbance.

CoV Real-Time RT-PCR LNA Assays.

Real-time RT-PCR LNA assays were performed using the One Step PrimeScript™ RT-PCR Kit (Perfect Real Time)® (TaKaRa, Japan). Each 20 μl reaction mixture contained lx One Step RT-PCR Buffer III®, 0.3 μM of each forward and reverse primer, 0.1 μM of probe, 2 U of TaKaRa Ex Taq HS®, 0.4 μl of PrimeScript RT enzyme Mix II®, 5.6 μl of nuclease-free water and 2 μl of RNA template. Amplification and detection were performed on the LightCycler 96® system (Roche Applied Science, Mannheim, Germany) or a Applied Biosystems 7500 Fast Dx® real-time PCR instrument (Life Technologies). Thermocycling conditions consisted of 5 minutes at 42° C. for reverse transcription, 10 seconds at 95° C. for inactivation of the RT enzyme, and 45 cycles of 5 seconds at 95° C. and 30 seconds at 56° C. for amplification. The MERS-CoV-upE assay was performed as described except that 5 μl of RNA template were used. A positive test result was defined as a well-defined exponential fluorescence curve that crossed threshold within 40 cycles. Negative and positive controls were included in all runs to monitor assay performance. The resulting assays showed excellent sensitivity across different strains of Coronavirus and no apparent crossreactivity between strains.

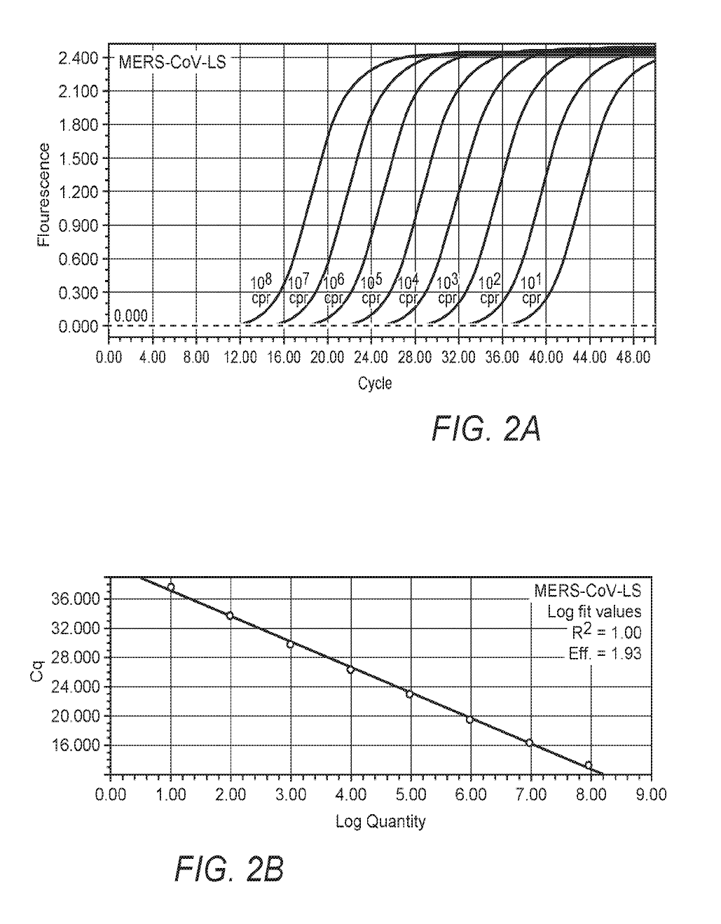

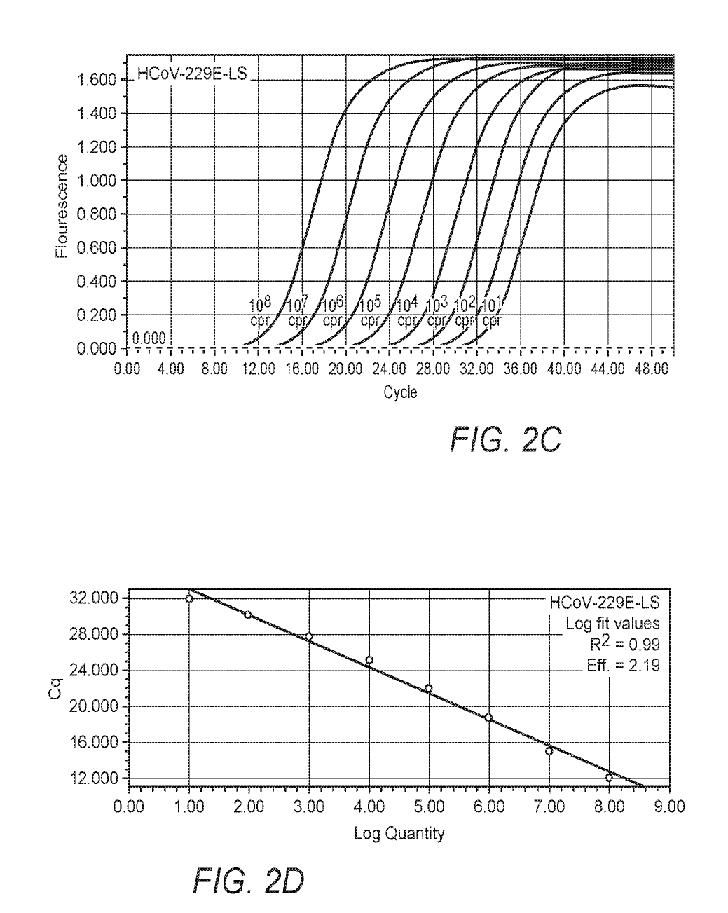

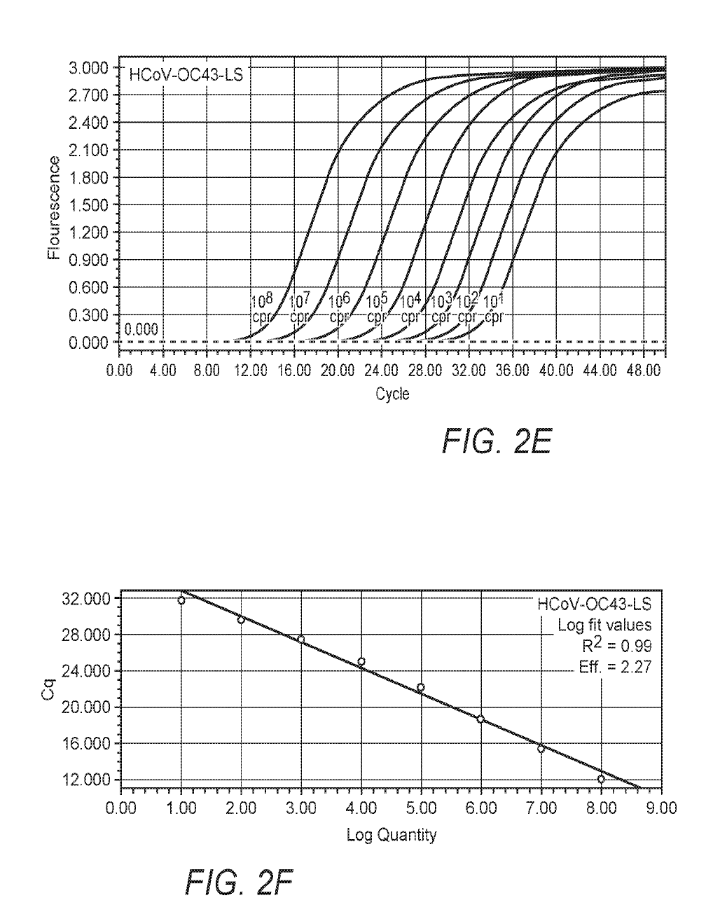

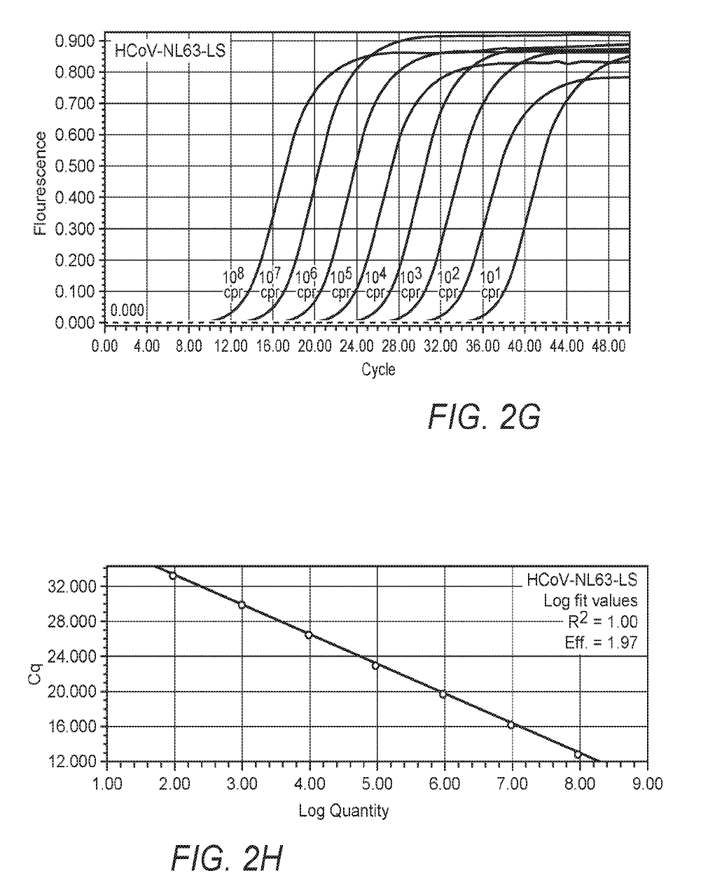

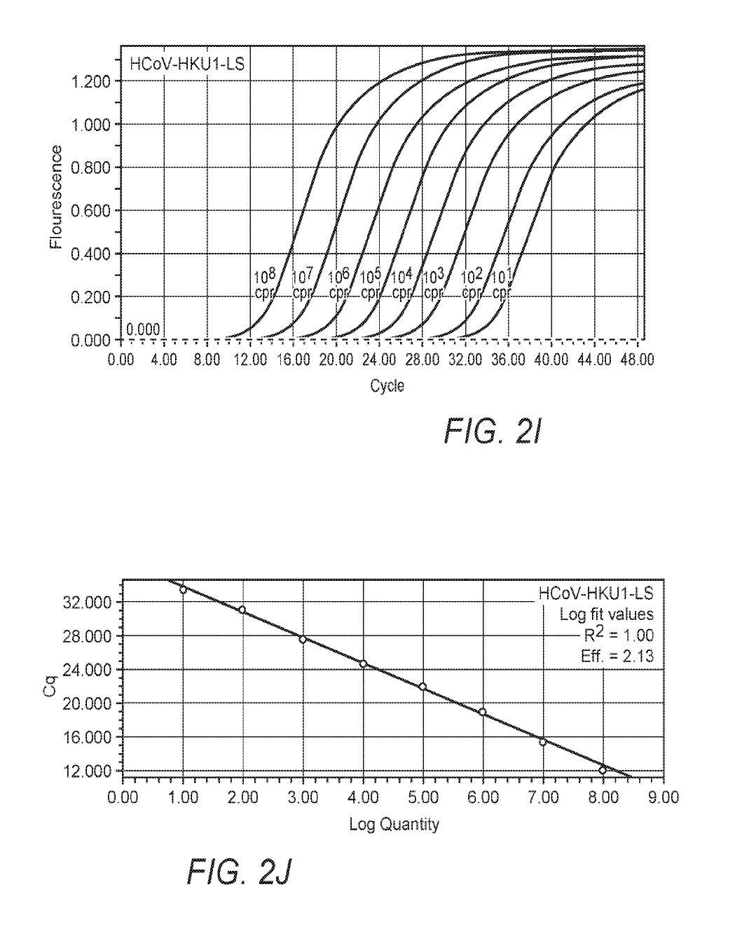

Results of typical RT-PCR assays performed using primer/probe sets of the inventive concept are shown in FIGS. 2A to 2I. FIG. 2A shows typical results of RT-PCR performed as described using a primer/probe set of the inventive concept that is directed towards MERS-CoV. Typical growth curves are evident for virus concentrations ranging from 10⁸ copies per reaction (cpr) to 10¹ (i.e. 10) cpr. FIG. 2B depicts a typical dose/response curve for the reactions of FIG. 2A, showing that RT-PCR results are highly linear. It is also evident that despite the relatively short length of the target sequence replication efficiency in the RT-PCR reaction is close to the theoretical limit. Such a dose/response curve can be used as a calibration curve for quantitation of the CoV virus. FIGS. 2C and 2D show corresponding results for RT-PCR performed using a primer/probe set for HCoV-229E. FIGS. 2D and 2E show corresponding results for RT-PCR performed using a primer/probe set for HCoV-OC43. FIGS. 2F and 2G show corresponding results for RT-PCR performed using a primer/probe set for HCoV-NL63. FIGS. 2H and 21 show corresponding results for RT-PCR performed using a primer/probe set for HCoV-HKU.1

Results of a typical study showing the limits of detection for different MERS-CoV and Human CoV strains are shown in Tables 6A and 6B.

TABLE 6A

| MERS-CoV-LS | | | | | MFRS-CoV-upE | | | | |
|---|---|---|---|---|---|---|---|---|---|
| Virus | | | | No. +/ | Virus | | | | No. +/ |
| quantity | Cq | | | no. | quantity | Cq | | | no. |
| (TCID₅₀/ml) | Test 1 | Test 2 | Test 3 | tested | (TCID₅₀/ml) | Test 1 | Test 2 | Test 3 | tested |
| 5.62 × 10⁴ | 13.82 | 14.18 | 13.88 | 3/3 | 5.62 × 10⁴ | 18.18 | 18.29 | 18.32 | 3/3 |
| 5.62 × 10³ | 18.26 | 18.39 | 18.51 | 3/3 | 5.62 × 10³ | 23.13 | 23.16 | 23.52 | 3/3 |
| 5.62 × 10² | 22.72 | 22.25 | 22.25 | 3/3 | 5 62 × 10² | 26.13 | 26.24 | 26.32 | 3/3 |
| 5.62 × 10¹ | 26.14 | 26.11 | 26.12 | 3/3 | 5.62 × 10¹ | 30.32 | 30.51 | 30.56 | 3/3 |
| 5.62 × 10⁰ | 29.31 | 29.35 | 29.40 | 3/3 | 5.62 × 10⁰ | 34.66 | 34.80 | 34.90 | 3/3 |
| 5.62 × 10⁻¹ | 33.17 | 33.20 | 33.20 | 3/3 | 5.62 × 10⁻¹ | 37.53 | 36.99 | 37.63 | 3/3 |
| 5.62 × 10⁻² | 37.32 | 35.56 | 36.69 | 3/3 | 5.62 × 10⁻² | 42.50 | 41.99 | — | 2/3 |
| 5.62 × 10⁻³ | — | — | — | 0/3 | 5.62 × 10⁻³ | 41.87 | — | — | 0/3 |
| 5.62 × 10⁻⁴ | — | — | — | 0/3 | 5.62 × 10⁻⁴ | — | — | — | 0/3 |

Abbreviations; +, positive; −, negative; Cq, quantification cycle; TCID₅₀, 50% tissue culture infective doses.

TABLE 6B

| HCoV-229E-LS | | | | | HCoV-OC43-LS | | | | |
|---|---|---|---|---|---|---|---|---|---|
| Virus | | | | No. | Virus | | | | No. |
| quantity | Cq | | | +/ no. | quanity | Cq | | | +/ no. |
| (TCID₅₀/ml) | Test 1 | Test 2 | Test 3 | tested | (TCID₅₀/ml) | Test 1 | Test 2 | Test 3 | tested |
| 5.00 × 10⁴ | 14.81 | 14.92 | 14.78 | 3/3 | 3.16 × 10³ | 14.37 | 14.38 | 14.44 | 3/3 |
| 5.00 × 10³ | 19.44 | 19.90 | 19.47 | 3/3 | 3.16 × 10² | 18.29 | 18.42 | 18.36 | 3/3 |
| 5.00 × 10² | 22.65 | 22.66 | 22.68 | 3/3 | 3.16 × 10¹ | 22.14 | 22.04 | 22.07 | 3/3 |
| 5.00 × 10¹ | 25.58 | 25.61 | 25.62 | 3/3 | 3.16 × 10⁰ | 25.37 | 25.33 | 25.30 | 3/3 |
| 5.00 × 10⁰ | 29.09 | 29.19 | 29.06 | 3/3 | 3.16 × 10⁻¹ | 28.78 | 28.71 | 28.73 | 3/3 |
| 5.00 × 10⁻¹ | 32.37 | 32.61 | 32.53 | 3/3 | 3.16 × 10⁻² | 32.62 | 32.35 | 32.17 | 3/3 |
| 5.00 × 10⁻² | 35.77 | 35.84 | 36.30 | 3/3 | 3.16 × 10⁻³ | 35.51 | 35.10 | 36.27 | 3/3 |
| 5.00 × 10⁻³ | — | — | — | 0/3 | 3.16 × 10⁻⁴ | — | — | — | 0/3 |
| 5.00 × 10⁻⁴ | — | — | — | 0/3 | 3.16 × 10⁻⁵ | — | — | — | 0/3 |

Abbreviations: +, positive; −, negative; Cq, quantification cycle; TCID₅₀, 50% tissue culture infective doses.

Results of crossreactivity studies are shown in Table 7.

TABLE 7

| | | MERS- | HCoV- | HCoV- | HCoV- | HCoV- |
|---|---|---|---|---|---|---|
| Virus | Source | CoV-LS | 229E-LS | OC43-LS | NL63-LS | HKU1-LS |
| MERS-CoV | Laboratory strain | + | − | − | − | − |
| HCoV-229E | Clinical specimen | − | + | − | − | − |
| HCoV-229E | Laboratory strain | − | + | − | − | − |
| HCoV-OC43 | Clinical specimen | − | − | + | − | − |
| HCoV-OC43 | Laboratory strain | − | − | + | − | − |
| HCoV-NL63 | Clinical specimen | − | − | − | + | − |
| HCoV-HKU1 | Clinical specimen | − | − | − | − | + |
| IAV (H1N1) | Clinical specimen | − | − | − | − | − |
| IAV (H1N1) | Laboratory strain | − | − | − | − | − |
| IAV (H3N2) | Clinical specimen | − | − | − | − | − |
| IAV (H3N2) | Laboratory strain | − | − | − | − | − |
| IBV | Clinical specimen | − | − | − | − | − |
| IBV | Laboratory strain | − | − | − | − | − |
| PIF 1 | Clinical specimen | − | − | − | − | − |
| PIF 1 | Laboratory strain | − | − | − | − | − |
| PIF 2 | Clinical specimen | − | − | − | − | − |
| PIF 2 | Laboratory strain | − | − | − | − | − |
| PIF 3 | Clinical specimen | − | − | − | − | − |
| PIF 3 | Laboratory strain | − | − | − | − | − |
| PIF 4 | Clinical specimen | − | − | − | − | − |
| PIF 4 | Laboratory strain | − | − | − | − | − |
| RV/EV | Clinical specimen | − | − | − | − | − |
| RV/EV | Laboratory strain | − | − | − | − | − |
| RSV | Clinical specimen | − | − | − | − | − |
| RSV | Laboratory strain | − | − | − | − | − |
| hMPV | Clinical specimen | − | − | − | − | − |
| hMPV | Laboratory strain | − | − | − | − | − |

Abbreviations: +, positive. −, negative. EV, enterovirus. HCoV, human coronavirus, IAV, influenza A virus: IBV, influenza B virus; hMPV, human metapneumovirus, MERS-CoV, Middle East respiratory syndrome coronavirus: PIF, parainfluenza virus, RSV, respiratory syncytial virus; RV, rhinovirus.

Confirmation of ResPlex II®-HCoV-Negative Samples Tested Positive by CoV Real-Time RT-PCR LNA Assays by Cloning and Sequencing.

The results of the CoV real-time RT-PCR LNA assays and ResPlex II® were compared. For specimens with discrepant results in the two assays, cloning and sequencing were performed to confirm the results. Each real-time RT-PCR product was cloned to confirm the identity. The real-time PCR product was purified by TaKaRa MiniBEST DNA Fragment Purification Kit Ver. 3.0® (TaKaRa, China), followed by cloning using TOPO TA Cloning® Kit Dual Promoter® (Invitrogen, USA) according to manufacturer's instructions. Plasmids of each real-time RT-PCR LNA assay-HCoV-positive but ResPlex II® HCoV-negative sample were purified using a QIAprep Spin Miniprep® Kit (Qiagen) and were sequenced with an ABI 3130×1 Genetic Analyzer® (Applied Biosystems). Typical results of testing of discrepant samples is shown in Table 8.

TABLE 8

| | | | Quantitative results of + |
|---|---|---|---|
| Overall CoV real-time RT-PCR LNA | | | samples by CoV real- |
| assays results | | | time RT-PCR LNA assays |
| Assay | − | + | (RNA copies/reaction) |
| MERS-CoV-LS | 180/180 | 0/180 | Not applicable |
| HCoV-229E-LS | 180/180 | 0/180 | Not applicable |
| HCoV-OC43-LS | 178/180 | 2/180 | Sample 1: 2.40 × 10² |
| | | | Sample 2: 1.84 × 10² |
| HCoV-NL63-LS | 178/180 | 2/180 | Sample 1: 2.29 × 10¹ |
| | | | Sample 2: 9.34 × 10¹ |
| HCoV-HKU1-LS | 180/180 | 0/180 | Not applicable |

What is claimed is:

1. A method of detecting a virus, comprising;
identifying a highly conserved nucleotide test sequence expressed at between 3% and 10% on infection of a suitable host cell by the virus;
synthesizing a probe sequence that is at least partially complementary to the test sequence;
hybridizing the probe sequence with the test sequence to form a hybridization complex; and
detecting the hybridization complex.

2. The method of claim 1, wherein the nucleotide test sequence is between 30 and 200 nucleotides in length.

3. The method of claim 1, wherein the nucleotide test sequence is between 60 and 90 nucleotides.

4. The method of claim 1, wherein the test sequence represents an untranslated region.

5. The method of claim 1, wherein the test sequence is expressed at greater than 5% on infection of a suitable host cell by the virus.

6. The method of claim 1, wherein the nucleotide test sequence comprises a leader sequence located at a 5′ untranslated region upstream to a transcription regulatory sequence.

7. The method of claim 1, wherein the probe sequence is identified in Table 1.

8. The method of claim 1, wherein the probe sequence comprises between 0.5% and 10% lack of complementarity to the test sequence.

9. The method of claim 1, wherein the probe sequence comprises a non-naturally occurring nucleotide.

10. The method of claim 9, wherein the non-naturally occurring nucleotide comprises an LNA.

11. The method of claim 1, wherein the probe sequence comprises a detectable tag.

12. The method of claim 11, wherein the detectable tag is selected from the group consisting of a fluorophore, a chromophore, a spin label, a radioactive isotope, an affinity epitope, and a mass tag.

13. The method of claim 1, wherein the virus is an RNA virus.

14. The method of claim 1, wherein the virus is selected from the group consisting of coronaviruses, Astroviridae, Caliciviridae, Picornaviridae, Flaviviridae, Retroviridae, Togaviridae, Arenaviridae, Bunyaviridae, Filoviridae, Orthomyxoviridae, Paramyxoviridae, Rhabdoviridae, and Reoviridae.

15. The method of claim 1, wherein the virus is a causative agent of SARS or MERS.

16. The method of claim 1, wherein the virus is an influenza virus.

17. The method of claim 1, wherein the hybridization complex comprises a polynucleotide duplex.

18. The method of claim 1, wherein the hybridization complex comprises a polynucleotide triplex.

19. The method of claim 1, wherein the detection step is performed without an exogenous polymerase driven amplification step.

20. The method of claim 1, wherein the detection step further includes an amplification step wherein at least a portion of the test sequence is replicated using an exogenous polymerase.

21. The method of claim 20, wherein the amplification step comprises PCR.

22. The method of claim 21, wherein the amplification step comprises RT-PCR.

23. The method of claim 21, wherein the amplification step comprises real time RT-PCR.

24. The method of claim 21, wherein the amplification step comprises nested PCR.

25. The method of claim 21, wherein the amplification step comprises a ligase chain reaction.

26. The method of claim 21, wherein the amplification step comprises a nucleic acid sequence based amplification.

27. The method of claim 19 or 20, further comprising contacting the test sequence with a microarray.

28. The method of claim 19 or 20, further comprising the step of obtaining a fluorescence measurement from the hybridization complex.

29. The method of claim 19 or 20, further comprising the step of contacting the hybridization complex with an affinity-directed molecule.

30. The method of claim 20, wherein the amplification step comprises an extension step, and wherein the extension step is characterized by having a temperature greater than 50° C.

31. A composition for detecting a virus comprising a probe sequence that is at least partially complementary to a highly conserved nucleotide test sequence expressed at between 3% and 10% on infection of a suitable host cell by the virus.

32. The composition of claim 31, wherein the nucleotide test sequence is between 30 and 200 nucleotides in length.

33. The composition of claim 31, wherein the nucleotide test sequence is between 60 and 90 nucleotides.

34. The composition of claim 31, wherein the test sequence represents an untranslated region.

35. The composition of claim 31, wherein the test sequence is expressed at greater than 5% on infection of a suitable host cell by the virus.

36. The composition of claim 31, wherein the nucleotide test sequence comprises a leader sequence located at a 5′ untranslated region upstream to a transcription regulatory sequence.

37. The composition of claim 31, wherein the probe sequence is identified in Table 1.

38. The composition of claim 31, wherein the probe sequence comprises between 0.5% and 10% lack of complementarity to the test sequence.

39. The composition of claim 31, wherein the probe sequence comprises a non-naturally occurring nucleotide.

40. The composition of claim 39, wherein the non-naturally occurring nucleotide comprises an LNA.

41. The composition of claim 31, wherein the probe sequence comprises a detectable tag.

42. The composition of claim 41, wherein the detectable tag is selected from the group consisting of a fluorophore, a chromophore, a spin label, a radioactive isotope, an affinity epitope, and a mass tag.

43. The composition of claim 31, wherein the virus is an RNA virus.

44. The composition of claim 31, wherein the virus is selected from the group consisting of coronaviruses, Astroviridae, Caliciviridae, Picornaviridae, Flaviviridae, Retroviridae, Togaviridae, Arenaviridae, Bunyaviridae, Filoviridae, Orthomyxoviridae, Paramyxoviridae, Rhabdoviridae, and Reoviridae.

45. The composition of claim 31, wherein the virus is a causative agent of SARS or MERS.

46. The composition of claim 31, wherein the virus is an influenza virus.

47. A kit for detecting a virus, comprising:
a probe sequence that is at least partially complementary to a highly conserved nucleotide test sequence expressed at between 3% and 10% on infection of a suitable host cell by the virus; and
instructions for use.

48. The kit of claim 47, wherein the nucleotide test sequence is between 30 and 200 nucleotides in length.

49. The kit of claim 47, wherein the nucleotide test sequence is between 60 and 90 nucleotides.

50. The kit of claim 47, wherein the test sequence represents an untranslated region.

51. The kit of claim 47, wherein the test sequence is expressed at greater than 5% on infection of a suitable host cell by the virus.

52. The kit of claim 47, wherein the nucleotide test sequence comprises a leader sequence located at a 5′ untranslated region upstream to a transcription regulatory sequence.

53. The kit of claim 47, wherein the probe sequence is identified in Table 1.

54. The kit of claim 47, wherein the probe sequence comprises between 0.5% and 10% lack of complementarity to the test sequence.

55. The kit of claim 47, wherein the probe sequence comprises a non-naturally occurring nucleotide.

56. The kit of claim 55, wherein the non-naturally occurring nucleotide comprises an LNA.

57. The kit of claim 47, wherein the probe sequence comprises a detectable tag.

58. The kit of claim 47, wherein the detectable tag is selected from the group consisting of a fluorophore, a chromophore, a spin label, a radioactive isotope, an affinity epitope, and a mass tag.

59. The kit of claim 47, wherein the virus is an RNA virus.

60. The kit of claim 47, wherein the virus is selected from the group consisting of coronaviruses, Astroviridae, Caliciviridae, Picornaviridae, Flaviviridae, Retroviridae, Togaviridae, Arenaviridae, Bunyaviridae, Filoviridae, Orthomyxoviridae, Paramyxoviridae, Rhabdoviridae, and Reoviridae.

61. The kit of claim 47, wherein the virus is a causative agent of SARS or MERS.

62. The kit of claim 47, wherein the virus is an influenza virus.

63. The kit of claim 47, further comprising an exogenous polymerase.

64. A method of improving the performance of an assay for an RNA virus comprising:
identifying a nucleotide test sequence expressed at between 3% and 10% on infection of a suitable host cell by the virus, wherein the test sequence is both untranslated and highly conserved;
synthesizing a probe sequence that is at least partially complementary to the test sequence;
hybridizing the probe sequence with the test sequence to form a hybridization complex; and
detecting the hybridization complex, wherein the method demonstrates at least one of enhanced specificity and enhanced sensitivity.

65. The method of claim 64, wherein the test sequence comprises a leader sequence located at a 5′ untranslated region upstream to a transcription regulatory sequence.

66. The method of claim 64, wherein the probe sequence comprises an LNA.

67. The method of claim 64, wherein the step of detecting the hybridization complex comprises RT-PCR.

68. The method of claim 67, wherein the RT-PCR utilizes a first primer and a second primer, wherein the first primer and the second primer represent a primer pair selected from sequences depicted in Table 1 or Table 2.

69. The method of claim 68, wherein the RT-PCR utilizes a third primer, wherein the third primer is selected from the sequences depicted in Table 1 or Table 2.

70. The method of claim 64, wherein the virus is selected from the group consisting of a coronavirus, an influenza A virus, and an influenza B virus.

71. A probe sequence for characterization of an RNA virus comprising a nucleotide sequence having at least partial complementarity to a highly conserved leader sequence located at a 5′ untranslated region upstream to a transcription regulatory sequence.

72. The probe sequence of claim 71, wherein the RNA virus is selected from the group consisting of a coronavirus, an influenza A virus, and an influenza B virus.

73. The probe sequence of claim 71, comprising a nucleotide sequence selected from sequences depicted in Table 1.

74. A primer sequence for characterization of an RNA virus, wherein the primer sequence is selected from sequences depicted in Table 1 or Table 2.

75. A primer pair for characterization of an RNA virus, wherein the primer pair comprises a first primer comprising a first nucleotide sequence and a second primer comprising a second nucleotide sequence, and wherein the first nucleotide sequence and the second nucleotide sequence are selected from the sequences depicted in Table 1 or Table 2.

76. A primer set for characterization of an RNA virus, wherein the primer set comprises:
a primer pair comprising a first primer having a first nucleotide sequence and a second primer having a second nucleotide sequence wherein the first nucleotide sequence and the second nucleotide sequence are selected from the sequences depicted as a primer pair in Table 1 or Table 2; and
an auxiliary primer having a third nucleotide sequence selected from the sequences depicted in Table 1 or Table 2,
wherein the third nucleotide sequence is distinct from the first nucleotide sequence and the second nucleotide sequence.

77. A probe sequence for characterization of an RNA virus, wherein the primer sequence is selected from sequences depicted in Table 1.
